(19) United States
(12) Patent Application Publication
(10) Pub. No.: US 2003/0049694 A1
(43) Pub. Date: Mar. 13, 2003

(54) Production of fusion proteins and use for identifying binding molecules

(76) Inventors: Chung-Hsiun Wu, Taipei (TW); Rong-Hwa Lin, Taipei (TW); Pei-Ling Hsu, Little Rock, AR (US)

Correspondence Address: FISH & RICHARDSON PC, 225 FRANKLIN ST, BOSTON, MA, 02110, US

(21) Appl. No.: 10/077,213

(22) Filed: Feb. 14, 2002

Related U.S. Application Data
(60) Provisional application No. 60/318,474, filed on Sep. 10, 2001.

(51) Int. Cl. G01N 33/00; G01N 33/567; C12Q 1/68

(52) U.S. Cl. 435/7.21; 435/6; 800/3

(57) ABSTRACT

The invention is directed to methods of producing a fusion protein by administering a nucleic acid encoding the fusion protein to an animal. Following the administration of the nucleic acid to the animal, the protein is produced in vivo and is isolated by removing a biological sample from the animal. These methods allow for the rapid and efficient production and isolation of a protein encoded by any nucleic acid sequence of interest. A fusion protein purified according to these methods can be used to screen for target binding molecules, such as antibodies, that bind to a protein sequence of interest.

### RELATED APPLICATIONS

This application claims priority from U.S. provisional application No. 60/318,474, filed on Sep. 10, 2001, the content of which is incorporated herein by reference.

### FIELD OF THE INVENTION

The invention relates to methods and compositions for producing proteins and identifying binding molecules.

### BACKGROUND OF THE INVENTION

Antibodies and other protein-specific binding ligands are valuable tools for both basic research and the diagnosis and treatment of various diseases. Protein-specific binding ligands allow for the precise identification and quantitation of a particular species of protein in a biological sample. In addition, the interaction between a protein and its binding ligand can modulate the activity or function of a protein. Modulation of the activity or function of a protein is particularly useful in treating a disease condition characterized by either excessive or insufficient activity of a given protein.

The identification of antibodies and other binding ligands typically involves screening methods that use a purified form of a protein. Purified proteins can be used in a wide variety of high throughout screening methodologies. These screening systems include the following: phage display libraries, single chain antibody libraries, nucleic acid-based binding ligands (such as oligo-ribonucleotides), and fibronectin-based binding ligands. The speed-limiting factor in the efficiency of many of these systems is the availability of protein molecules to be screened.

### SUMMARY OF THE INVENTION

The invention is directed to methods of producing a fusion protein by administering a nucleic acid encoding the fusion protein to an animal. According to these methods, any nucleic acid sequence of interest can be inserted into a hybrid nucleic acid construct and administered to an animal. Following the administration of the nucleic acid to the animal, the fusion protein is produced in vivo and is isolated following the removal of a biological sample from the animal. These methods allow for the rapid and efficient production and isolation of a fusion protein encoded by any nucleic acid sequence of interest.

A fusion protein purified according to these methods can be used to screen for target binding molecules, such as antibodies, that bind to a protein sequence of interest. In general, the methods include an initial immobilization of the fusion protein on a solid surface. This captured fusion protein is then used to screen for target binding molecules. This invention thus permits the high-throughput screening of antibodies or other ligands directed against any protein sequence, without the need to synthesize the protein sequence in vitro. In addition, antibodies directed against an amino acid sequence can readily be produced by administering to an animal a nucleic acid encoding a fusion protein containing a specific amino acid sequence, to thereby elicit an immune response against the specific amino acid sequence.

In one aspect, the invention includes a method of isolating a target binding molecule by performing the following steps: (1) administering to a mammal a nucleic acid encoding a fusion protein and expressing the fusion protein in the mammal, wherein the fusion protein contains a first amino acid sequence and a second amino acid sequence, and wherein the second amino acid sequence contains a first member of a specific binding pair; (2) removing from the mammal a biological sample that contains the fusion protein; (3) binding a second member of the specific binding pair to the fusion protein via the first member of the specific binding pair; (4) providing a solution containing a target binding molecule, wherein the target binding molecule binds to the first amino acid sequence of the fusion protein; and (5) isolating the target binding molecule by means of its binding to the fusion protein.

In one embodiment, the first member of the specific binding pair is a peptide of at least five amino acids in length. For example, the first member of the specific binding pair can be an Fc domain of an immunoglobulin.

The biological sample used in the method can include, for example, serum or a tissue lysate.

In one embodiment, the second member of the specific binding pair is an antibody, e.g., a monoclonal antibody.

The target binding molecule can be, for example, a protein, a nucleic acid, or a small molecule. In one embodiment, the target binding molecule is an antibody. An antibody can be prepared in a variety of ways, either in vitro or in vivo. For example, an antibody can be prepared in a mammal by immunizing the mammal with a nucleic acid construct encoding the fusion protein.

The method can also include an additional step of administering a protease inhibitor to the mammal before removing the biological sample from the mammal.

The method can also include an additional step of immobilizing the fusion protein.

In another aspect, the invention features a method of preparing a purified fusion protein by performing the following steps: (1) administering to a mammal a nucleic acid encoding a fusion protein and expressing the fusion protein in the mammal, wherein the fusion protein contains a first amino acid sequence and a second amino acid sequence, and wherein the second amino acid sequence contains a first member of a specific binding pair; (2) removing from the mammal a biological sample that contains the fusion protein; (3) binding a second member of the specific binding pair to the fusion protein via the first member of the specific binding pair; and (4) removing components of the biological sample that are not bound to the second member of the specific binding pair, to thereby provide a purified fusion protein.

The method can include an additional step of cleaving the first amino acid sequence from the second amino acid sequence.

In one embodiment, the first member of the specific binding pair is a peptide of at least five amino acids in length. For example, the first member of the specific binding pair can be an Fe domain of an immunoglobulin.

The biological sample used in the method can include, for example, serum or a tissue lysate.

In one embodiment, the second member of the specific binding pair is an antibody, e.g., a monoclonal antibody.

The method can include an additional step of immobilizing the fusion protein.

An advantage of the invention is that it provides rapid and efficient methods for the production of large amounts of a protein or portion thereof in its native conformation. The purified protein can then be used in methods such as screening for target binding molecules. These production methods avoid the time-consuming, expensive, and laborious tasks associated with the in vitro production and purification of proteins.

Another advantage of the invention is that it avoids several of the difficulties associated with recombinant protein production in bacteria. Mammalian proteins expressed in bacteria lack post-translational modification and frequently do not possess their native conformation. In addition, some proteins cannot even be translated in some bacteria because some nucleic acid constructs contain codons that are rarely used in bacteria.

Numerous screening systems have been developed to search for molecules that interact with proteins of interest. The speed-limiting factor in many of these systems is the availability of protein molecules to be screened. In many cases, the screening machinery remains idle while a protein of interest is produced according to traditional methods. The present invention can thus complement these screening systems by rapidly and efficiently providing a protein of interest.

Unless otherwise defined, all technical and scientific terms used herein have the same meaning as commonly understood by those of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of a conflict in terminology, the present specification will control. In addition, the described materials and methods are illustrative only and are not intended to be limiting.

Other features and advantages of the invention will be apparent from the following detailed description and the claims.

### BRIEF DESCRIPTION OF THE DRAWINGS

FIG. 1 depicts the detection of a Cr1-tagged IL-5-fusion protein produced by the administration to an animal of a nucleic acid encoding the fusion protein. The detection was carried out using an IL-5-specific antibody.

FIG. 2 depicts the detection of a Cr1-tagged EGFP-MEN1-fusion protein produced by the administration to an animal of a nucleic acid encoding the fusion protein. The detection was carried out using an EGFP-specific antibody.

### DETAILED DESCRIPTION

The invention is directed to methods of preparing a protein of interest by administration of a nucleic acid encoding the protein to an animal. The protein is produced within the animal and subsequently immobilized by the use of an affinity reagent that specifically binds to the protein (e.g., binds to a portion of the protein engineered to contain an affinity tag). Once immobilized, screens can be carried out to identify molecules, such as antibodies or any other binding molecules, that bind to the protein. In addition, the binding titer of antibodies or ligands in biological fluids, culture medium, or in any buffer can be quantitated.

Nucleic Acid Constructs

The invention encompasses the use of a variety of nucleic acid constructs encoding fusion proteins. The fusion proteins encoded by the nucleic acids described herein contain at least two heterologous amino acid sequences, designated a first amino acid sequence and a second amino acid sequence. Because these two amino acid sequences are heterologous, the sequence of the fusion protein thus differs from that of a naturally occurring protein.

The identity of the first amino acid sequence is essentially unlimited. The first amino acid sequence is generally a focus of the methods of the invention, in that a variety of applications can be carried out on this sequence, including but not limited to screening methods to identify target binding molecules such as antibodies that bind to the sequence. In one example, the first amino acid sequence is identical to all or a portion of a naturally occurring protein. Preferably, the first amino acid sequence is at least 6, 10, 25, 50, 100, or 200 amino acids in length. In another example, the first amino acid sequence contains an epitope that can be recognized by a target binding molecule, e.g., an antibody.

The second amino acid sequence of the fusion protein contains a sequence that permits the immobilization and/or purification of the fusion protein. Therefore, the second amino acid sequence contains a first member of a specific binding pair that can bind to a second member of the specific binding pair. In one example of a specific binding pair, the first member of the pair is a stretch of amino acids that is bound by the second member of the pair, e.g., an antibody such as a monoclonal antibody. One embodiment of such a binding pair is an immunoglobulin Fc constant region (first member of the specific binding pair) and an anti-immunoglobulin Fc constant region antibody (second member of the specific binding pair). In another example of a specific binding pair, the first member of the binding pair is a stretch of amino acids that is bound by a non-protein affinity agent. An example of such a specific binding pair is a poly-histidine tag (first member of the specific binding pair) and a nickel affinity reagent (second member of the specific binding pair). For example, a tag of 6-10 histidine residues, when included in a fusion protein, can be used to purify the fusion protein by nickel affinity chromatography (see, e.g., Copeland et al. (1996) Nature 379:162-165).

Amino acid sequences in addition to the first and second amino acid sequences described herein can be included in the fusion protein. For example, a targeting sequence can be used that directs trafficking of the fusion protein (e.g., the targeting sequence can direct the fusion protein to a specific cellular compartment, to the plasma membrane, or can direct secretion of the protein). In one example, the fusion protein contains a signal peptide sequence. A signal peptide sequence refers to a short (usually about 15-60 amino acids), continuous stretch of amino acids located at the amino-terminus of nascent secreted and membrane-bound polypeptides, which directs their delivery to various locations. Signal sequences usually contain a hydrophobic core of about 4-15 amino acids, which is often immediately preceded by a basic amino acid. At the carboxyl-terminal end of the signal peptide there-are a pair of small, uncharged amino acids separated by a single intervening amino acid that defines the signal peptide cleavage site (see, e.g., von Heijne (1990) J. Membrane Biol. 195-201).

A fusion protein can optionally contain other amino acid sequences, in addition to the first and second amino acid sequences, that carry out substantially the same functions as the first and/or second amino acid sequences. For example, a fusion protein can contain an additional member of a specific binding pair and/or an additional sequence that can be bound by a target binding molecule. Thus, a single fusion protein can contain, in addition to the second amino acid sequence, sequences derived from multiple different proteins and can thus be used to generate antibodies against the different sequences and/or to screen for target binding molecules directed to these different sequences.

A fusion protein can optionally contain an amino acid sequence that permits the cleavage of the fusion protein. For example, a protein cleavage site can be placed between the first and second amino acid sequences. Such a cleavage site can permit the separation of the first amino acid sequence from the second amino acid sequence following purification of the fusion protein. For example, the fusion protein can contain a sequence of amino acids that includes a specific recognition site for enzymatic cleavage which does not occur elsewhere in the molecule. Examples of useful sites are the sequences that are recognized and cleaved by blood factor Xa or enterokinase. A cleavage enzyme can thus by chosen by reference to its recognition sequence.

A fusion protein can optionally contain one or more linker sequences. A linker sequence can be used to provide spacing and/or orientation to the first and/or second amino acid sequences and promote the biological functioning of each of the sequences. A linker sequence generally separates two sequences by a distance sufficient to ensure that each folds into its secondary structure. A linker sequence length of about 20 amino acids can be used to provide a suitable separation of functional protein domains, although longer or shorter linker sequences can also be used (e.g., lengths of 3 to 100 amino acids). Amino acid sequences useful as linkers include, but are not limited to, (SerGly₄)_{y} (SEQ ID NO:1) wherein y is at least 2, or Gly₄SerGly₅Ser (SEQ ID NO:2). A preferred linker sequence has the formula (SerGly₄)₄ (SEQ ID NO:3). Another preferred linker has the sequence ((Ser₄ Gly)₃-Ser-Pro) (SEQ ID NO:4). Alternatively, a fusion protein can lack linker sequences.

Nucleic acid constructs can be prepared using conventional molecular biology techniques. A nucleic acid can be operatively linked to regulatory elements that direct expression of the coding sequence. These regulatory elements include, but are not limited to, inducible and non-inducible promoters, enhancers, and other elements which are known to those skilled in the art and which drive or otherwise regulate gene expression. Such regulatory elements include but are not limited to the cytomegalovirus (CMV) immediate early gene, a retroviral LTR promoter, and the early or late promoters of SV40. In a preferred embodiment, expression of the nucleic acid is under the control of a human CMV immediate early promoter.

Nucleic acid constructs are preferably incorporated into a vector, e.g., an expression vector such as a plasmid or a viral vector. Specific initiation signals may also be required for efficient translation of nucleic acid molecules. These translational control signals include the ATG initiation codon and adjacent sequences (e.g., sequences that conform with the Kozak consensus sequence). Furthermore, the initiation codon must be in phase with the reading frame of the desired coding sequence to ensure translation of the entire insert. Translational control signals and initiation codons can be of a variety of origins, both natural and synthetic. The efficiency of expression may be enhanced by the inclusion of appropriate transcription enhancer elements, transcription terminators, or introns (see Bittner et al. (1987) Methods Enzymol 153:516).

In vivo Production of Fusion Proteins

A fusion protein is produced by administering a nucleic acid described herein to an animal, e.g., a mammal such as a mouse rat, goat, rabbit, or human. Following administration of the nucleic acid, translation (as well as transcription if the nucleic acid is a DNA sequence) occurs in vivo and the fusion protein is thus produced within the animal. The nucleic acid can be administered to the animal by a variety of routes, e.g., intravenously, intramuscularly, intraarterially, intradermally, intraperitoneally, intranasally, or subcutaneously.

The nucleic acid can be “naked” or complexed with a delivery vehicle. Examples of useful delivery vehicles include but are not limited to microparticles, liposomes, ISCOMS, or any other suitable delivery vehicles. For a description of useful methods of gene transfer, see, e.g., Templeton et al. (1997) Nature Biotechnology 15:647-652 and U.S. Pat. No. 6,214,804.

One particularly useful means of obtaining high level expression of a nucleic acid in an animal is by use of a hydrodynamics-based transfection procedure (see, e.g., Liu et al. (1999) Gene Therapy 6:1258-1266; Zhang et al. (1999) Human Gene Therapy 10:1735-1737; Zhang et al. (2000) Gene Therapy 7:1344-1349; He et al. (2000) Human Gene Therapy 11:547-554). According to these methods, a large volume of liquid containing a nucleic acid of interest is injected rapidly into an animal. This method can result in gene expression within 8-24 hours as well as extended gene expression over days or weeks. For example, the injection into the tail vain of a mouse of approximately 1.5 to 2.5 ml of a solution containing about 5-25 ug of plasmid DNA at a rate of approximately 0.3 ml/second can result in expression of the fusion protein encoded by the nucleic acid. If the nucleic acid construct encodes a sequence that directs secretion of the fusion protein, e.g., a signal peptide sequence, then significant amounts of the fusion protein are secreted into the blood of the animal.

The production of a fusion protein in an animal, as described herein, can be accompanied by the administration to the animal of a protease inhibitor or a plurality of different protease inhibitors. Examples include inhibitors of serine, cysteine, or aspartic proteases as well as inhibitors of aminopeptidases. The administration of protease inhibitors to the animal can increase the yield of functional fusion protein isolated from the animal. The protease inhibitors can be administered before, during, or after the administration of the nucleic acid to the animal.

Once the fusion protein is produced within the animal, a biological sample containing the fusion protein can be removed from the animal. As described above for the hydrodynamics-based transfection procedure (e.g., as carried out together with the in vivo administration of protease inhibitors), significant amounts of the fusion protein are produced within about 8-24 hours following the administration of the nucleic acid. The animal may need to be sacrificed, depending upon the nature of the sample to be removed. For example, the fusion protein can be found within the blood or a solid organ including but not limited to the liver, kidney, spleen, heart, and/or lung. Following the removal of a solid organ, a tissue lysate may optionally be prepared to facilitate isolation and processing of the fusion protein.

Immobilization of Fusion Proteins

Following the removal of a biological sample containing a fusion protein from an animal, an in vitro method is carried out to immobilize the fusion protein. As described herein, the fusion protein contains a first member of a specific binding pair. The biological sample is brought into contact with a second member of the specific binding pair, and the fusion protein contained within the sample is thereby bound to the second member. Unbound material contained within the biological sample can be removed by washing. Methods of immobilizing proteins by means of interactions between members of specific binding pairs are well known to those of skill in the art. As described herein, the second member of the specific binding pair can include proteins such as antibodies as well as non-protein affinity reagents such as nickel (for use in purifying proteins containing a poly histidine tag).

In one example, a fusion protein is captured by using antibodies (specific to the second amino acid sequence of the fusion protein) coated on a solid phase surface. Monoclonal or polyclonal antibodies specific for the second amino acid sequence can be immobilized on a suitable solid phase surface by various methods known to those in the art. In a preferred embodiment of the present invention, human immunoglobulin Fc constant gamma-1 (Cr1) specific antibodies are used. The solid phase surface is not limited to any particular form and includes plastic tubes, microtiter plates, beads such as cellulose beads and agarose beads, latex particles, magnetic particles, paper, and dipsticks. Methods for immobilizing antibodies include passive absorption, covalent linkage, and physical trapping. The fusion protein is captured on the solid phase surface by incubation of the fusion protein-containing biological sample (e.g., sera or tissue lysates) with second amino acid sequence-specific antibodies pre-immobilized on the surface. Unbound materials can be removed by washing.

The immobilized fusion protein can be used for a variety of purposes. For example, the fusion protein can be used to screen for molecules that bind to the first amino acid sequence portion of the protein. In addition, the fusion protein can be used to screen for molecules that inhibit the interaction between the first amino acid sequence and a naturally occurring ligand of the first amino acid sequence. These screening methods are discussed in further detail in subsequent sections. In another example, the fusion protein can be purified (by the removal of unbound material contained in the biological sample) and used for a variety of purposes, e.g., to analyze the activity and/or structure of the protein. The fusion protein or a portion thereof can optionally be purified to homogeneity following the immobilization and washing steps. As described herein, the fusion protein can optionally contain a sequence, e.g. a sequence located between the first and second amino acid sequences, that permits cleavage of the fusion protein and the isolation of the first amino acid sequence.

Generation of Antibodies

For the immunization process, a nucleic acid construct as described herein is administered to an animal, e.g., a mammal such as a mouse, rat, goat, rabbit, or human. This administration results in the production of the fusion protein within the animal and the generation of an immune response directed against the first amino acid sequence of the fusion protein. This immune response can be exploited to prepare antibodies, either polyclonal or monoclonal, that recognize the first amino acid sequence. The nucleic acid is delivered by a route and in a dosage that is sufficient to induce a humoral immune response. For example, the nucleic acid can be administered intraperitoneally, intravenously, intramuscularly, intraarterially, intradermally, intranasally, or subcutaneously.

The following is an illustrative embodiment of a method of preparing a monoclonal antibody using a nucleic acid described herein. A nucleic acid construct is combined with a mixture of lipids in a 1:10 ratio and injected intraperitoneally into a mouse at a dose of 50 ug/ml of DNA per mouse. A boost injection with the same dose through the same route is performed at day 10 and a test serum is collected at day 21. The titer of the polyclonal serum is determined using an assay described herein. If the titer of the polyclonal serum is satisfactory at day 21, then a third boost is performed at day 22, and a cell fusion experiment to produce hybridoma can be performed at day 29. Monoclonal antibodies can be produced by applying, for example, the well known cell fusion method of Kohler and Milstein ((1976) European Journal of Immunology 6:511-519) to produce hybridomas secreting the desired antibodies in the culture supernatants. After obtaining a homogenous population of cells, which is usually done by limiting dilution cloning, the antibody producing hybridomas are grown in vitro or in vivo and the specificity of a monoclonal antibody can then be characterized by the screening methods of the invention.

Screening for Target Binding Molecules

As described herein, a fusion protein can be produced within an animal and subsequently immobilized via an interaction between members of a specific binding pair. These rapid and efficient methods of producing and immobilizing a protein containing any sequence of interest (the first amino acid sequence) can be used to screen for molecules, referred to herein as target binding molecules, that bind to the first amino acid sequence and/or inhibit the ability of a ligand to bind to the first amino acid sequence. These methods are particularly well adapted for high throughput screening for antibodies and other ligands that specifically bind to the first amino acid sequence portion of the fusion protein. In addition to or as part of a screening system, the methods of the invention include the identification, quantitation, and/or purification of target binding molecules. For example, a target binding molecule can be identified by contacting a collection of candidate target binding molecules (e.g., a library) to a fusion protein containing the first amino acid sequence, and subsequently determining chemical or physical properties of the bound molecule or molecules (e.g., by determining the amino acid sequence of a bound peptide or the chemical structure of a bound member of a synthetic chemical library).

Following the immobilization of a fusion protein on a surface, the fusion protein is contacted with a sample containing at least one candidate target binding molecule. A candidate target binding molecule can be of any class of molecule that can bind to a protein sequence. Examples of target binding molecules include but are not limited to proteins (used interchangeably with the terms polypeptides or peptides) antibodies and fragments thereof, ribonucleic acids, small molecules, ribozymes, antisense oligonucleotides, deoxyribonucleic acids, and other organic compounds. Target binding molecules may include peptides such as, for example, soluble peptides, including but not limited to members of random peptide libraries and combinatorial chemistry-derived molecular libraries made of D- and/or L configuration amino acids, phosphopeptides (including, but not limited to, members of random or partially degenerate, directed phosphopeptide libraries; see, e.g., Songyang et al. (1993) Cell 72:767), antibodies (including, but not limited to, polyclonal, monoclonal, humanized, anti-idiotypic, chimeric or single chain antibodies, and Fab, F(ab′)₂ and Fab expression library fragments, phage display libraries, and epitope-binding fragments thereof), aptamers (nucleic acid molecules that have a tertiary structure permitting them to specifically bind to protein ligands (see, e.g., Osborne et al. (1997) Curr. Opin. Chem Biol. 1:5-9)), fibronectin-based binding ligands, and small organic or inorganic molecules.

A target binding molecule can also be a naturally occurring ligand of the first amino acid sequence. For example, following immobilization of a fusion protein, the fusion protein can be contacted with a biological sample that contains a naturally occurring ligand of the first amino acid sequence. Following binding and washing steps, the identity of the bound ligand can be determined by methods well known to those of skill in the art.

In one method of the invention, test sera containing target protein specific antibodies is produced by immunizing an animal with a nucleic acid construct described herein. Following the production of antibodies within the animal, test serum can be collected and screened as described herein.

Screening assays can be conducted in a variety of ways. For example, one method to conduct such an assay involves anchoring the fusion protein onto a solid phase (by the binding of the members of a specific binding pair) and detecting fusion protein/target binding molecule complexes anchored on the solid phase at the end of the reaction. In one embodiment of such a method, the fusion protein is anchored onto a solid surface, and the test compound, which is not anchored, is labeled, either directly or indirectly.

As described herein for the immobilization of the fusion protein, a wide variety of solid phases can be used for the immobilization and screening processes. For example, microtiter plates can conveniently be utilized as the solid phase. The second member of the specific binding pair, e.g., a monoclonal antibody, can be immobilized to the solid phase by a non-covalent or covalent attachment. Non-covalent attachment can be accomplished by simply coating the solid surface with a solution containing the second member of the specific binding pair. The surfaces can be prepared in advance and stored.

In order to conduct the assay, the target binding molecule is added to the surface containing the fusion protein. After the reaction is complete, unbound components are removed (e.g., by washing) under conditions such that any complexes formed will remain immobilized on the solid surface. The detection of complexes anchored on the solid surface can be accomplished in a number of ways. Where the previously non-immobilized target binding molecule is pre-labeled, the detection of label immobilized on the surface indicates that complexes were formed. Where the previously non-immobilized target binding molecule is not pre-labeled, an indirect label can be used to detect complexes anchored on the surface, e.g., using a labeled antibody specific for the previously non-immobilized target binding molecule (the antibody, in turn, may be directly labeled or indirectly labeled with a labeled anti-Ig antibody).

Alternatively, a reaction can be conducted in a liquid phase, the bound products separated from unbound components, and complexes detected, e.g., using an immobilized antibody specific for the fusion protein to anchor any complexes formed in solution, and a labeled antibody specific for the target binding molecule of the possible complex to detect anchored complexes.

The incubation times and temperatures for these procedures are not narrowly critical. For example, incubation times can range from 10 minutes to 48 hours, and preferably these are carried out from 1 to 2 hours. The incubation temperatures can range from 4° C. to 37° C. Preferred incubation temperatures are 20° C. to 37° C.

The fusion protein is captured by incubation of fusion protein-containing sera or tissue lysates with specific antibodies pre-immobilized on a surface. After removal of unbound non-specific substances, the testing antibodies are incubated with the solid phase and target protein specific antibodies are quantitatively detected with a binding partner that is labeled or tagged with a signal generating marker. For example, if the testing sera is derived from mouse, the binding partner can be signal generating marker-labeled antibodies specific to mouse IgG antibodies.

In this one embodiment of the present invention, the binding partner can be labeled or tagged with a variety of signal generating markers. These include (but are not limited to) enzymes, chemiluminescent compounds, fluorescent labels, dyes, radioisotopic labels, enzyme cofactors, and biotin. The chemical linkage of this binding partner to a signal-generating marker can be accomplished by a variety of methods known to those in the art.

The following are examples of the practice of the invention. They are not to be construed as limiting the scope of the invention in any way.

### EXAMPLES

### Example 1

### In vivo Production of a Human-IL-5-Immunoglobulin Fc Constant Region Fusion Protein and Detection with an IL-5-Specific Antibody

A nucleic acid sequence encoding human IL-5 was fused to the human immunoglobulin Fc constant region gamma 1 (Cr1). Ten ug of a DNA construct encoding the IL-5-Cr1 fusion protein was diluted in 2 ml of phosphate buffered saline (PBS) and injected intravenously into a mouse within a period of 10 seconds.

The liver was harvested from the mouse 20 hours after injection of the DNA. One hour before harvesting the liver, 100 ul of a protease inhibitor cocktail (Sigma, P8340) was injected intravenously into the mouse. The liver was homogenized and the IL-5-Cr1 fusion protein contained in the lysate was captured on a microtiter plate pre-coated with a Cr1-specific antibody. The goat anti-human IgG Fc gamma-1 antibody (layer 1; a capture antibody) was immobilized on a microtiter plate (NUNC-Immuno™ plate, MaxiSorp™ Surface) by an overnight incubation of the antibody at a concentration of 2 ug/ml.

The IL-5-Cr1 fusion protein (layer 2) was bound to layer 1 by incubating a liver lysate (diluted in 1% BSA and 10% normal goat serum containing blocking buffer) in the wells of the microtiter plate at room temperature for 2 hours. Liver lysate which contained a different fusion protein (EGFP-MEN-Cr1) was used in parallel wells as a control for background subtraction. After four washes to remove unbound material, the plates were incubated with an IL-5-specific mouse monoclonal antibody (R&D Systems Inc., Minneapolis, Minn.) diluted in normal mouse sera. The amount of IL-5-specific antibody (layer 3) bound to the plates was determined by adding the following: biotinylated goat anti-mouse IgG (1/5000) (layer 4); streptavidin-alkaline phosphatase enzyme conjugates (1/10000) (layer 5); and 1 mg/ml p-nitrophenyl phosphate (enzyme substrate), followed by an incubation at 4° C. for 15 hours. The presence of alkaline phosphatase conjugated streptavidin was detected by measuring the absorption at OD 405 nm, after adding the enzyme substrate.

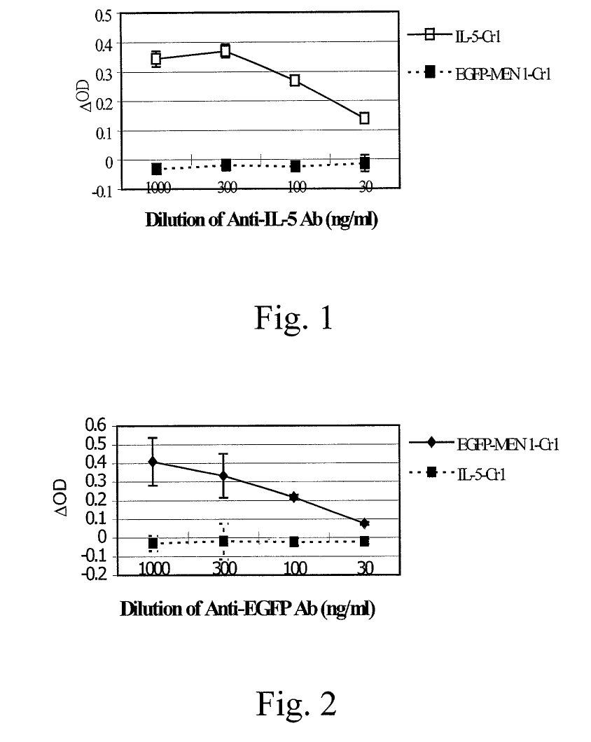

In FIG. 1, the X axis depicts the dilution factor of the positive test serum (layer 3), and the Y axis depicts delta OD (calculated as the OD derived from fusion protein-containing liver lysates minus the OD derived from normal liver lysates). FIG. 1 shows that the assay detects the presence of target protein-specific antibodies on the microtiter plate and that the delta OD for each dilution of the positive serum displays a linear correlation against the dilution factor. Each point in the figure is an average of triplicate wells and the figure is representative of at least two independent experiments.

### Example 2

### In vivo Production of a EGFP-MEN1-Immunoglobulin Fc Constant Region Fusion Protein and Detection with an EGFP-Specific Antibody

A nucleic acid sequence encoding EGFP-MEN1 was fused to the human immunoglobulin Fc constant region gamma 1 (Cr1). Ten ug of a DNA construct encoding the EGFP-MEN1-Cr1 fusion protein was diluted in 2 ml of phosphate buffered saline (PBS) and injected intravenously into a mouse within a period of 10 seconds.

The liver was harvested from the mouse 20 hours after injection of the DNA. One hour before harvesting the liver, 100 ul of a protease inhibitor cocktail (Sigma, P8340) was injected intravenously into the mouse. The liver was homogenized and the EGFP-MEN1-Cr1 fusion protein contained in the lysate was captured on a microtiter plate pre-coated with a Cr1-specific antibody. The goat anti-human IgG Fc gamma-1 antibody (layer 1; a capture antibody) was immobilized on a microtiter plate (NUNC-Immuno™ plate, MaxiSorp™ Surface) by an overnight incubation of the antibody at a concentration of 2 ug/ml.

The EGFP-MEN1-Cr1 fusion protein (layer 2) was bound to layer 1 by incubating a liver lysate (diluted in 1% BSA and 10% normal goat serum containing blocking buffer) in the wells of the microtiter plate at room temperature for 2 hours. Liver lysate which contained a different fusion protein (IL-5-Cr1) was used in parallel wells as a control for background subtraction. After four washes to remove unbound material, the plates were incubated with an EGFP-specific mouse monoclonal antibody (Clontech Laboratories, Inc., Palo Alto, Calif.) diluted in normal mouse sera. The amount of EGFP-specific antibody (layer 3) bound to the plates was determined by adding the following: biotinylated goat anti-mouse IgG (1/5000) (layer 4); streptavidin-alkaline phosphatase enzyme conjugates (1/10000) (layer 5); and 1 mg/ml p-nitrophenyl phosphate (enzyme substrate), followed by an incubation at 4° C. for 15 hours. The presence of alkaline phosphatase conjugated streptavidin was detected by measuring the absorption at OD 405 nm, after adding the enzyme substrate.

In FIG. 2, the X axis depicts the dilution factor of the positive test serum (layer 3), and the Y axis depicts delta OD (calculated as the OD derived from fusion protein-containing liver lysates minus the OD derived from normal liver lysates). FIG. 2 shows that the assay detects the presence of target protein-specific antibodies on the microtiter plate and that the delta OD for each dilution of the positive serum displays a linear correlation against the dilution factor. Each point in the figure is an average of triplicate wells and the figure is representative of at least two independent experiments.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method of isolating a target binding molecule, the method comprising:
administering to a mammal a nucleic acid encoding a fusion protein and expressing the fusion protein in the mammal, wherein the fusion protein contains a first amino acid sequence and a second amino acid sequence, and wherein the second amino acid sequence contains a first member of a specific binding pair;
removing from the mammal a biological sample that contains the fusion protein;
binding a second member of the specific binding pair to the fusion protein via the first member of the specific binding pair;
providing a solution containing a target binding molecule, wherein the target binding molecule binds to the first amino acid sequence of the fusion protein; and
isolating the target binding molecule by means of its binding to the fusion protein.

2. The method of claim 1, wherein the first member of the specific binding pair is an Fc domain of an immunoglobulin.

3. The method of claim 1, wherein the biological sample is serum.

4. The method of claim 1, wherein the biological sample is tissue lysate.

5. The method of claim 1, wherein the second member of the specific binding pair is an antibody.

6. The method of claim 5, wherein the antibody is a monoclonal antibody.

7. The method of claim 1, wherein the target binding molecule is a protein.

8. The method of claim 1, wherein the target binding molecule is an antibody.

9. The method of claim 8, wherein the antibody is prepared in an animal by immunizing the animal with a nucleic acid construct encoding the fusion protein.

10. The method of claim 1, further comprising administering a protease inhibitor to the mammal before removing the biological sample from the mammal.

11. The method of claim 2, wherein the target binding molecule is an antibody.

12. The method of claim 11, wherein the antibody is prepared in an animal by immunizing the animal with a nucleic acid construct encoding the fusion protein.

13. The method of claim 1, wherein the target binding molecule is a nucleic acid.

14. The method of claim 2, wherein the target binding molecule is a nucleic acid.

15. The method of claim 1, wherein the target binding molecule is a small molecule.

16. The method of claim 2, wherein the target binding molecule is a small molecule.

17. The method of claim 1, further comprising immobilizing the fusion protein.

18. The method of claim 2, further comprising immobilizing the fusion protein.

19. The method of claim 1, wherein the first member of the specific binding pair is a peptide of at least five amino acids in length.

20. A method of preparing a purified fusion protein, the method comprising:
administering to a mammal a nucleic acid encoding a fusion protein and expressing the fusion protein in the mammal, wherein the fusion protein contains a first amino acid sequence and a second amino acid sequence, and wherein the second amino acid sequence contains a first member of a specific binding pair;
removing from the mammal a biological sample that contains the fusion protein;
binding a second member of the specific binding pair to the fusion protein via the first member of the specific binding pair; and
removing components of the biological sample that are not bound to the second member of the specific binding pair, to thereby provide a purified fusion protein.

21. The method of claim 20, further comprising cleaving the first amino acid sequence from the second amino acid sequence.

22. The method of claim 20, wherein the first member of the specific binding pair is an Fc domain of an immunoglobulin.

23. The method of claim 20, wherein the biological sample is serum.

24. The method of claim 20, wherein the biological sample is tissue lysate.

25. The method of claim 20, wherein the second member of the specific binding pair is an antibody.

26. The method of claim 25, wherein the antibody is a monoclonal antibody.

27. The method of claim 22, wherein the second member of the specific binding pair is an antibody.

28. The method of claim 27, wherein the antibody is a monoclonal antibody.

29. The method of claim 20, further comprising immobilizing the fusion protein.

30. The method of claim 21, further comprising immobilizing the fusion protein.

31. The method of claim 22, further comprising immobilizing the fusion protein.

32. T method of claim 20, wherein the first member of the specific binding pair is a peptide of at least five amino acids in length.
